(19) United States
(12) Patent Application Publication
(10) Pub. No.: US 2018/0042628 A1
(43) Pub. Date: Feb. 15, 2018

(54) Methods And Devices To Remove Thromboembolic Material From Blood Vessels

(71) Applicant: Justin Panian, San Francisco, CA (US)

(72) Inventor: Justin Panian, San Francisco, CA (US)

(21) Appl. No.: 15/621,860

(22) Filed: Jun. 13, 2017

Related U.S. Application Data
(60) Provisional application No. 62/494,520, filed on Aug. 10, 2016.

(51) Int. Cl. A61B 17/22

(52) U.S. Cl. CPC A61B 17/22031; A61B 2017/22034; A61B 2017/22035; A61B 2017/22038; A61B 2017/22094; A61B 2017/22051

(57) ABSTRACT

A guard device for removal of thromboembolic material from a blood vessel includes a placement catheter having at least one axial lumen, a shield device having a pusher wire with an expandable braid assembly attached to its distal end and deliverable through the lumen of the placement catheter. The expandable braid assembly has a distal end with a distal tip at the distal end, where the distal tip prevents the distal end of the expandable braid assembly from fully expanding when deployed from the placement catheter. The expandable braid assembly has a diameter that is at least 1.5 times larger in its expanded position than in its collapsed configuration when inside the placement catheter.

### FIELD OF THE INVENTION

Relevant Art
U.S. Patènt Documents

| | |
|---|---|
| 4,347,846 September 1982 Dormia | 4,706,671 November 1987 Weinrib |
| 4,776,337 October 1988 Palmaz | 4,886,062 December 1989 Wiktor |
| 5,011,488 April 1991 Ginsburg | 5,192,286 March 1993 Phan et al. |
| 5,354,308 October 1994 Simon et al. | 5,370,653 December 1994 Cragg |
| 5,370,683 December 1994 Fontaine | 5,456,667 October 1995 Ham et al. |
| 5,496,365 March 1996 Sgro | 5,643,312 July 1997 Fischell et al. |
| 5,667,486 September 1997 Mikulich et al. | 5,681,335 October 1997 Serra et al. |
| 5,795,331 August 1998 Cragg et al. | 5,800,519 September 1998 Sandock |
| 5,800,520 September 1998 Fogarty et al. | 5,800,525 September 1998 Bachinski et al. |
| 5,810,872 September 1998 Kanesaka et al. | 5,827,321 October 1998 Roubin et al. |
| 5,836,966 November 1998 St. Germain | 5,843,117 December 1998 Alt et al. |
| 5,855,600 January 1999 Alt | 5,876,449 March 1999 Starck et al. |
| 5,879,370 March 1999 Fischell et al. | 5,895,398 April 1999 Wensel et al. |
| 5,895,406 April 1999 Gray et al. | 5,911,754 June 1999 Kanesaka et al. |
| 5,913,895 June 1999 Burpee et al. | 5,968,088 October 1999 Hansen et al. |
| 5,972,018 October 1999 Israel et al. | 5,984,929 November 1999 Bashiri et al. |
| 6,027,526 February 2000 Limon et al. | 6,030,397 February 2000 Monetti et al. |
| 6,042,597 March 2000 Kveen et al. | 6,059,822 May 2000 Kanesaka et al. |
| 6,066,149 May 2000 Samson et al. | 6,066,158 May 2000 Engelson et al. |
| 6,106,548 August 2000 Roubin et al. | 6,146,403 November 2000 St. Germain |
| 6,200,335 March 2001 Igaki | 6,206,911 March 2001 Milo |
| 6,217,608 April 2001 Penn et al. | 6,273,910 August 2001 Limon |
| 6,309,414 October 2001 Rolando et al. | 6,350,271 February 2002 Kurz et al. |
| 6,398,805 June 2002 Alt | 6,402,431 June 2002 Nish |
| 6,402,771 June 2002 Palmer et al. | 6,409,754 June 2002 Smith et al. |
| 6,423,091 July 2002 Hojeibane | 6,468,301 October 2002 Amplatz et al. |
| 6,468,302 October 2002 Cox et al. | 6,475,236 November 2002 Roubin et al. |
| 6,478,816 November 2002 Kveen et al. | 6,482,217 November 2002 Pintor et al. |
| 6,488,703 December 2002 Kveen et al. | 6,491,719 December 2002 Fogarty et al. |
| 6,514,273 February 2003 Voss et al. | 6,551,342 April 2003 Shen et al. |
| 6,575,995 June 2003 Huter et al. | 6,582,447 June 2003 Patel et al. |
| 6,626,936 September 2003 Stinson | 6,641,590 November 2003 Palmer et al. |
| 6,660,021 December 2003 Palmer et al. | 6,679,893 January 2004 Tran |
| 6,692,504 February 2004 Kurz et al. | 6,706,054 March 2004 Wessman et al. |
| 6,716,240 April 2004 Fischell et al. | 6,818,013 November 2004 Mitelberg et al. |
| 6,818,613 November 2004 Sharma et al. | 6,881,222 April 2005 White et al. |
| 6,949,120 September 2005 Kveen et al. | 6,960,228 November 2005 Mitelberg et al. |
| 7,008,434 March 2006 Kurz et al. | 7,037,321 May 2006 Sachdeva |
| 7,037,331 May 2006 Mitelberg et al. | 7,081,130 July 2006 Jang |
| 7,108,714 September 2006 Becker | 7,195,648 March 2007 Jones et al. |
| 7,291,166 November 2007 Cheng et al. | 7,300,458 November 2007 Henkes et al. |
| 7,311,726 December 2007 Mitelberg et al. | 7,316,692 January 2008 Huffmaster |
| 7,485,130 February 2009 St. Germain | 7,651,513 January 2010 Teoh et al. |
| 7,655,033 February 2010 Feller, III et al. | 7,678,119 March 2010 Little et al. |
| 7,811,300 October 2010 Feller, III et al. | 7,875,044 January 2011 Feller, III et al. |
| 7,887,560 February 2011 Kusleika | 8,062,347 November 2011 Tenne |
| 8,357,178 January 2013 Grandfield et al. | 8,357,179 January 2013 Grandfield et al. |
| 8,529,596 September 2013 Grandfield et al. | 8,795,317 August 2014 Grandfield et al. |
| 8,795,345 August 2014 Grandfield et al. | 9,044,263 Jun. 2, 2015 Grandfield et al |
| 9,072,537 Jul. 7, 2015 Grandfield et al. | 9,119,656 Sep. 1, 2015 Bose et al. |
| 9,149,609 Oct. 6, 2015 Ansel et al. | |

US Published Applications

| | |
|---|---|
| 2001/0047200 November 2001 White et al. | 2003/0004567 January 2003 Boyle et al. |
| 2003/0100917 May 2003 Boyle et al. | 2003/0116751 June 2003 Elman |
| 2003/0176914 September 2003 Rabkin et al. | 2003/0199921 October 2003 Palmer et al. |
| 2004/0068314 April 2004 Jones et al. | 2004/0199175 October 2004 Jaeger et al. |
| 2004/0236368 November 2004 McGuckin, Jr | 2005/0209678 September 2005 Henkes |
| 2005/0267491 December 2005 Kellett et al. | 2006/0116751 June 2006 Bayle et al. |
| 2006/0265048 November 2006 Cheng et al. | 2006/0287701 December 2006 Pal |
| 2007/0038178 February 2007 Kusleika | 2007/0190866 August 2007 Zart et al. |
| 2007/0191866 August 2007 Palmer et al. | 2007/0198051 August 2007 Clubb et al. |
| 2007/0225739 September 2007 Pintor et al. | 2007/0280367 December 2007 Nakao et al. |
| 2007/0288054 December 2007 Tanaka et al. | 2008/0125855 May 2008 Henkes et al. |
| 2008/0208244 August 2008 Boylan et al. | 2008/0262487 October 2008 Wensel et al. |
| 2009/0105722 April 2009 Fulkerson et al. | 2009/0105737 April 2009 Fulkerson et al. |
| 2010/0100106 April 2010 Ferrera | 2010/0114135 May 2010 Wilson et al. |
| 2010/0161034 June 2010 Leanna et al. | 2010/0174309 July 2010 Fulkerson et al. |
| 2010/0318097 December 2010 Ferrera et al. | 2010/0331853 December 2010 Garcia et al. |
| 2011/0009875 January 2011 Grandfield | 2011/0009940 January 2011 Grandfield |
| 2011/0009941 January 2011 Grandfield | 2011/0009950 January 2011 Grandfield |
| 2011/0130784 June 2011 Kusleika | 2011/0184456 July 2011 Grandfield et al. |
| 2011/0196414 August 2011 Porter et al. | 2012/0123466 May 2012 Porter et al. |
| 2012/0215250 August 2012 Grandfield | 2014/0046338 February 2014 Grandfield |
| 2014/0277082 September 2014 Janardham et al. | 2015/0238207 Aug. 27, 2015 Cox et al. |
| 2015/0327875 November 2015 Look et al | 2015/0327977 November 2015 Zaver et al. |

Foreign Patent Documents

| | |
|---|---|
| 2003254553 February 2004 AU | 2492978 January 2004 CA |
| 4032759 April 1992 DE | 19834956 May 1999 DE |
| 10233085 January 2004 DE | 10301850 July 2004 DE |
| 0897698 February 1999 EP | 0914807 May 1999 EP |
| 0916362 May 1999 EP | 1266640 December 2002 EP |
| 1266640 December 2002 EP | 1266640 December 2002 EP |
| 1362564 November 2003 EP | 1534178 October 2007 EP |
| 1351626 February 2008 EP | 1542617 January 2011 EP |
| 1542617 January 2011 EP | 2463592 March 2010 GB |
| 62049841 March 1987 JP | 7124251 May 1995 JP |
| 2001511030 August 2001 JP | 2003512887 April 2003 JP |
| 2004536666 December 2004 JP | 2005532887 November 2005 JP |
| 2006521865 September 2006 JP | 2008512207 April 2008 JP |
| 2008519668 June 2008 JP | 2008522757 July 2008 JP |
| 2010264261 November 2010 JP | WO9704711 February 1997 WO |
| WO9725000 July 1997 WO | WO9832412 July 1998 WO |
| WO0132099 May 2001 WO | WO0145592 June 2001 WO |
| WO03011188 February 2003 WO | WO2004006804 January 2004 WO |
| WO2004008991 January 2004 WO | WO2004093696 November 2004 WO |
| WO2006029321 March 2006 WO | WO2006053270 May 2006 WO |
| WO2006063222 June 2006 WO | WO2008063156 May 2008 WO |
| WO2010010545 January 2010 WO | |

The present invention relates to methods and devices for removing thromboembolic materials from blood vessels, including cerebral arteries, and for the treatment of Acute Ischemic Stroke.

### DESCRIPTION OF THE PRIOR ART

Stroke is a leading cause of death and disability in the US with over 700,000 people suffering a major stroke and over 150,000 deaths each year. This tragic situation is expected to get worse as the “baby boomer” population reaches advanced age, and with increasing population obesity, which are two main contributing factors leading to stroke. Of those who survive a stroke, approximately 90% will suffer deficit including long-term impairment of movement, sensation, memory or reasoning, ranging from mild to severe. The total cost to the US healthcare system is estimated to be over $60 billion per year. Strokes may be caused by a rupture of a cerebral artery (“hemorrhagic stroke”) or a blockage in a cerebral artery due to a thromboembolism (“ischemic stroke”). A thromboembolism is a detached blood clot that travels through the bloodstream and lodges so as to obstruct or occlude a blood vessel. Between the two types of strokes, ischemic stroke comprises the larger problem, with over 600,000 people in the US suffering from ischemic stroke per year.

Ischemic stroke may be treated using a pharmacological elimination of the thromboembolism and/or by mechanical removal of the thromboembolism. Pharmacological elimination may be accomplished via the administration of thombolytics (e.g. streptokinase, urokinase, tissue plasminogen activator (TPA)), and/or anticoagulant drugs (e.g., heparin, warfarin) designed to dissolve and prevent further growth of the thromboembolism. Pharmacologic treatment is non-invasive and generally effective in dissolving the thromboembolism. However, significant drawbacks exist with the use of pharmacologic treatment. One such drawback is the relatively long amount of time required for the thrombolytics and/or anticoagulants to take effect and restore blood flow. Given the time-critical nature of treating ischemic stroke, any added time is potentially devastating. Another significant drawback is the increased risk of potential bleeding or hemorrhage elsewhere in the body due to the thombolytics and/or anticoagulants.

Mechanical removal of thromboembolic material for the treatment of ischemic stroke has been attempted using a variety of catheter-based transluminal interventional techniques. Most of such interventional techniques involve deploying a helical member into a thromboembolism in an effort to ensnare or envelope the thromboembolism so it can be removed from the patient. Although an improvement over pharmacologic treatments for ischemic stroke, such clot retrieval systems have only slightly increased clot removal success due to thromboembolic material slipping past or becoming dislodged by the removal devices. The dislodgement of thromboembolic material may lead to an additional stroke in the same artery or a connecting artery.

Another interventional technique involves deploying a basket or net structure distally (or downstream) from the thromboembolism in an effort to ensnare or envelope the thromboembolism so it can be removed from the patient. While such an approach overcomes the drawbacks of pharmacologic treatment, it requires extended manipulations of the basket or net and increases the danger of damaging the vessel and the potential of dislodging clot mass that also may lead to distal flow of thromboembolic material.

Latest interventional techniques for treating ischemic stroke involve advancing a suction catheter to the thromboembolism with the goal of removing it via aspiration (i.e. negative pressure Although generally safe, removal via aspiration is only effective with relatively soft thromboembolic material. When facing a more organized clot mass, such aspiration catheters tend to get clogged and require removal of the catheter, catheter cleaning, and repeating aspiration of remaining clots. Such techniques also carry clot dislodgement and additional stroke risk.

Interventional techniques described in the prior art are sub-optimal for treating ischemic stroke. The present invention is intended for improvement of the weaknesses of the prior art by providing blood clot removal devices and methods capable of efficient removal of the blood clots from large and small cerebral vessels. Particularly, the present invention provides methods and devices to remove emboli from cerebral vessels that minimize separation of the blood clot mass to be removed, which could escape and travel distally.

### SUMMARY OF THE INVENTION

The devices and methods of the present invention are suitable for shielding and removal of thromboembolic material from the human cerebral arteries and treatment of Acute Ischemic Stroke. The devices may also be deployed and used in other endovascular locations and ducts throughout the body.

The clot or thromboembolic material removal devices of the present invention typically comprise a guard assembly device having a placement catheter, a shielding device including a pusher wire and at least one expandable braid attached to the pusher wire and deliverable through the placement catheter, and an aspiration catheter and aspiration pump with clot collecting accessories.

When the expandable braid is released outside of the placement catheter beyond the location of the thromboembolic material, it expands outwardly against the blood vessel wall forming a shield that prevents thromboembolic mass or any of its parts from distal flow. Then, the aspiration catheter is activated to remove thromboembolic material.

In a preferred embodiment of the present invention, a guard device for thromboembolic material removal from a blood vessel is provided and comprises a placement catheter having at least one axial lumen, and a shield device comprising a pusher wire with an expandable braid assembly attached to its distal end and deliverable through the lumen of the placement catheter. The expandable braid assembly is movable during deployment from a first delivery position to a second placement position, where in the first delivery position the expandable braid assembly is in an unexpanded position inside the placement catheter having a nominal first diameter, and where in the second position the expandable braid assembly is in a radially expanded position having a second nominal diameter greater than the first nominal diameter when deployed within the vasculature of a patient.

In another embodiment, the shield device with expandable braid assembly traverses concomitant bends as the placement catheter when delivered through the placement catheter to the thromboembolic material location.

In another embodiment, the expandable braid assembly has a distal end/tip that prevents a very distal end of the expandable braid from fully expanding when deployed from the placement catheter. The tip may be made of one of the following materials: metal, polymer, rubber, adhesive or any combination thereof.

In yet another embodiment, the expandable braid assembly has a preset expanded transverse shape including: circular, non-circular or a combination of both, and has a distal end/tip and a proximal end/tip that prevents both ends from fully expanding when deployed from the placement catheter.

In another embodiment, the proximal end of the expandable braid assembly expands to a cylindrical shape with a fully open proximal end.

In yet another embodiment the expandable braid assembly includes at least one radiopaque marker positioned on the distal end, on the proximal end, or on both ends. Such radiopaque marker may be positioned inside the expandable braid assembly on the outside surface of the expandable braid assembly, or on both locations. The radiopaque marker may be included in a radiopaque solder. A radiopaque component may also be included within the expandable braid assembly.

In another embodiment, the expandable braid assembly is at least as large as the treatment area and has a diameter that is at least 1.5 times larger in its expanded configuration versus its collapsed configuration when inside the placement catheter. Such braid may be formed from a plurality of strands of Nitinol wire having an outside diameter between 0.0005 inches and 0.002 inches and a pore size formed between strands in the expanded configuration of less than about 0.5 square mm.

In yet another embodiment the expandable braid assembly may be formed from a plurality of strands of Nitinol wire having multiple wire strands of equal dimensions, or of different dimensions, braided into the tubular shape using circular wire, oval wire, flat wire or any other suitable wire configuration or combinations.

In another embodiment, the expandable braid assembly may be also made of Nitinol/Platinum composite.

In another embodiment, the expanded braid assembly is configured to have a pre-set expanded diameter of the cross-sectional shape including one of the following configurations: circular shape, non-circular shape or a combination of both.

In another embodiment, the expandable braid assembly comprises between 8-72 strands made of a monofilament wire having a braid angle of 40 degrees or less in the collapsed configuration inside the placement catheter, the expandable braid assembly is configured to have an expanded braid angle between about 90-150 degrees and the expanded braid assembly outside diameter is between about 1 mm to about 8.0 mm.

In yet another embodiment, a friction reduction means is located on the surface of the expandable braid assembly to improve ease of deployment and retrieval out of and into the placement catheter.

In yet another embodiment, the expandable braid assembly is made of a monofilament wire having a closed pitch of about 5-50 picks per inch in the collapsed configuration inside the placement catheter and 20-100 picks per inch in the expanded configuration.

In another embodiment the expandable braid assembly has dimensional and material characteristics that result in higher radial forces on the proximal end of the braid. The expandable braid has the radial force exerted by the expandable braid assembly being close to zero when the expandable braid assembly is expanded.

In yet another embodiment the expandable braid assembly comprises one or more undulations including but not limited to twists, bends, folds, waves, changes in cross sectional profile, or other.

In another embodiment at least one elongate constraining member is extended at least partially through the expandable braid assembly. Such constraining member may enhance the radiopacity of the expandable braid assembly by having a radiopaque composition.

In another preferred embodiment, a guard device for thromboembolic material removal from a blood vessel is provided which comprises a placement catheter having at least one lumen extended longitudinally, and a shield device comprising a pusher wire with expandable braid assembly having at least two subsequent braids attached to its distal end of the pusher wire and slidable in the lumen of the placement catheter. The expandable braid assembly is movable during deployment from a first delivery position to a second placement position, where in the first delivery position the expandable braid assembly is in an unexpanded position inside the placement catheter having a nominal first diameter, and where in the second position the expandable braid assembly is in a radially expanded position having a second nominal diameter greater than the first nominal diameter when deployed within the vasculature of a patient.

In another embodiment, the shield device with the expandable braid assembly traverses concomitant bends as the placement catheter when delivered through the placement catheter to the thromboembolic material location.

In another embodiment, the dual expandable braid assembly comprises the following configurations: a larger distal expandable braid and smaller proximal expandable braid connected together, or one continuous braid having two different dimensions.

In yet another preferred embodiment, a guard device for thromboembolic material removal from a blood vessel is provided which comprises a placement catheter having at least one lumen extended longitudinally, and a shield device comprising a pusher wire with an expandable braid assembly having an inner expandable braid and an outer expandable braid attached to the pusher wire and deliverable through the lumen of the placement catheter. The expandable braid assembly is movable during deployment from a first delivery position to a second placement position, where in the first delivery position the expandable braid assembly is in an unexpanded position inside the placement catheter having a nominal first diameter, and where in the second position the expandable braid assembly is in a radially expanded position having a second nominal diameter greater than the first nominal diameter when deployed within the vasculature of a patient.

In another embodiment, the shield device with the expandable braid assembly traverses concomitant bends as the placement catheter when delivered through the placement catheter to the thromboembolic material location.

In another embodiment, the expandable braid assembly is configured with the proximal end of the outer expandable braid open-ended, the inner expandable braid located inside the outer expandable braid, the distal end of the inner expandable braid attached to the distal end of the outer expendable braid, and the pusher wire attached to the proximal end of the inner expandable braid.

In another embodiment, the inner expandable braid and the outer expandable braid are configured with the proximal end of the outer expandable braid open-ended, the distal end of the outer expandable braid having a tip, the inner expandable braid having a distal tip connected to the distal tip of the outer expandable braid, and a proximal tip attached to the pushing member.

In yet another embodiment, the outer larger expandable braid and smaller inner expandable braids are configured with the inner and outer expandable braids about the same length in the expanded configuration, the inner expandable braid being shorter than the outer expandable braid in the expanded configuration, or the inner expandable braid being longer than the outer expandable braid in the expanded configuration.

In another preferred embodiment of the present invention, a method for removing a thromboembolic material from a blood vessel is provided. The method provides a guard device including a placement catheter having an axial lumen and a shield device having a pusher wire attached to an expandable braid assembly and deliverable through the lumen of the placement catheter. The distal end of the placement catheter is passed through the thromboembolic material in the blood vessel, then the shield device is advanced through the placement catheter. The expandable braid is deployed such that the expandable braid is located distally beyond the thromboembolic material. Next, the placement catheter is withdrawn outside the blood vessel, and an aspiration catheter is introduced over the pusher wire to the proximal end of the thromboembolic material. The thromboembolic material is aspirated outside the blood vessel, the guard device and the aspiration catheter are removed outside the blood vessel.

In yet another embodiment, the expandable braid assembly is movable during deployment from a first delivery position to a second placement position. In the first delivery position, the expandable braid assembly is in an unexpanded position inside the placement catheter having a nominal first diameter. In the second position, the expandable braid assembly is in a radially expanded position having a second nominal diameter greater than the first nominal diameter when deployed within the vasculature of a patient.

In another embodiment, the proximal end of the expandable braid assembly expands inside the vessel to a cylindrical shape with a fully open proximal end and wherein the proximal open end of the expanded braid has one of the following dimensions: smaller than the size of the vessel, equal to the vessel size, or larger than the vessel size. In each case, a deployed expandable braid assembly provides distal protection to prevent thromboembolic material from moving distally.

In yet another embodiment, the expandable braid assembly exerts radial forces to the vessel wall when expanded to a conforming shape as the blood vessel, or a larger size than the vessel size.

In another embodiment, the placement catheter is positioned inside the blood vessel using a guidewire.

In yet another embodiment, the expandable braid assembly may be repositioned after deployment.

In another embodiment, the placement catheter is introduced to the treatment area through the aspiration catheter.

In yet another embodiment, the expandable braid assembly expanded inside the vessel is configured to have a pre-set expanded shape, including one of the following configurations: circular shape, non-circular shape or a combination of both.

In another embodiment the placement catheter has a sufficient flexibility to navigate the vasculature of the patient. The placement catheter comprises a proximal end, a distal end and an inner lumen, with the inner lumen having a diameter sufficient to receive the expandable braid in its unexpanded position and for advancing the unexpanded braid from the proximal end to the distal end of the placement catheter, and the expandable braid is configured to permit proximal retraction of the braid into the lumen of the placement catheter when the braid is partially or fully deployed outside the distal end of the placement catheter.

In yet another embodiment, the expandable braid assembly is retrieved inside the aspiration catheter to exert pressure against the thromboembolic material in a radially inward direction to facilitate removal of the thromboembolic material and to prevent the aspiration catheter from clogging.

In another embodiment, the aspiration catheter is pushed against the proximal end of the expanded braid assembly to exert pressure against the thromboembolic material in a radially forward direction to facilitate removal of the thromboembolic material and to prevent the aspiration catheter from clogging.

In yet another embodiment, the aspiration catheter and/or the shield device are repositioned during removal of thromboembolic material.

In another embodiment, the shield device is retracted into the aspiration catheter and removed from the blood vessel upon removal of thromboembolic material.

In yet another embodiment, a method for removing thromboembolic material from a blood vessel includes inserting a placement catheter through the thromboembolic material, and introducing a shield device having a pusher wire attached to an expandable braid assembly having at least two expandable braids into the placement catheter, wherein the proximal expandable braid is smaller and distal expandable braid is larger when in expanded position. The method also includes deploying the expandable braid assembly from the placement catheter distally to the thromboembolic material location, wherein the deployed dual expandable braid assembly provides distal protection to prevent embolic material from moving distally. The method also includes removing the placement catheter, introducing an aspiration catheter, aspirating thromboembolic material outside the body, and removing the aspiration catheter and shield device outside the body.

In another embodiment, the dual in line expandable braid assembly is retracted at least partially into the aspiration catheter when the aspiration catheter clogs.

In accordance with another embodiment of the present invention, the shield device comprises a pusher wire and an expandable braid assembly having at least two expandable braid sections: a larger distal braid section attached to the distal end of the pusher wire and a smaller proximal braid section movable at least partially along the pusher wire. When the shield device is in the expanded configuration, the distal braid provides distal protection to prevent embolic material from moving distally, while the proximal braid provides a separator or plunger that can be moved inside the aspiration catheter in case when the aspiration catheter is clogged.

In accordance with a further embodiment, the shield device comprises an internal stopper that prevents the proximal braid from collapsing when retrieved into the aspiration catheter when it is being unclogged.

In yet another embodiment, a distal portion of the aspiration catheter is advanced against the proximal expandable braid to exert pressure against the thromboembolic material in a radially forward direction to facilitate clot removal.

In another embodiment of the present invention, a method for removing thromboembolic material from a blood vessel includes inserting a placement catheter through the thromboembolic material, introducing a shield device into the placement catheter, deploying the shield device expandable braid at least partially from the placement catheter distally beyond the thromboembolic material location, wherein deployed shield device provides distal protection to prevent embolic material from moving distally, removing the placement catheter, introducing an aspiration catheter, aspirating thromboembolic material outside the body, and removing the aspiration catheter and shield device outside the body. A variety of shield devices may be used as described in the present invention disclosure.

In yet another embodiment, the shield device may be rotated during blood clot removal to cause the blood clot to wobble, shake or be disrupted to further expedite clot removal. The shield device may be rotated clockwise and/or anti-clockwise while pulling back the shield device into the aspiration catheter.

In another embodiment, maximum aspiration pressure is applied instantaneously to clots to avoid clogging of the aspiration catheter.

In yet another embodiment, the shield device engages into the clot material to be removed. When the shield device is rotated the clot material rotates as well. Also, when the shield device is repositioned longitudinally, the clot material moves too.

In another embodiment, the shield device comprises the expandable braid and an expandable separator. The expandable separator provides means to unplug a clogged aspiration catheter, while the expandable braid prevents particles or emboli from moving distally.

Several alternative shield devices are described in the present invention describing methods and devices to remove thromboembolic material from blood vessels. All shield devices of the present invention are designated to perform two fundamental functions: (i) distal protection and separation, or (ii) plunger function to facilitate unclogging aspiration catheters. Both these functions are performed by shield device regardless of its structure, such as a single device, assembly device, or combined device. Furthermore, other structures of the shield device may include but are not limited to: non-braids, expandable clot pullers and distal protection devices, and dual balloon device, among others

### DRAWINGS DESCRIPTION

FIG. 1—Illustrates a schematic view of a guard device for the removal of thromboembolic material from a blood vessel with a shield device inside the placement catheter (expandable braid assembly in a collapsed configuration).

FIG. 2—Illustrates a schematic view of the same guard device as in FIG. 1 with a shield device having dual inner and outer braids deployed outside the placement catheter (expandable braid assembly in expanded/released configuration).

FIG. 3—Illustrates a schematic view of an alternative guard device with the shield device having dual in line expandable braids deployed outside the placement catheter (expandable braid in expanded/released configuration).

FIG. 4—Shows another alternative version of the guard device with a shield device made of a single expandable braid assembly with tips located on both ends in an expanded configuration.

FIG. 5—Shows another alternative guard device with a shield device comprising a pusher wire and a pusher tube attached to the shield device.

FIGS. 6A, 6B, 6C—Show alternative versions of the guard device that includes a retrieval sleeve.

FIG. 7—Shows another alternative version of the guard device having a shield device comprising an expandable braid and an expandable separator.

FIG. 8—Shows the placement catheter positioned proximally at the thromboembolic material location and the guidewire across the thromboembolic material.

FIG. 9—Shows the placement catheter positioned across the thromboembolic material location with the shield device inside the placement catheter.

FIG. 10—Shows the shield device deployed distally to the thromboembolic material location and the placement catheter removed.

FIG. 11—Shows the aspiration catheter placed over the pusher wire of the shield device at the location of the thromboembolic material.

FIG. 12—Shows the aspiration catheter clogged and the shield device pulled back into the aspiration catheter to unclog the aspiration catheter.

FIG. 13—Shows the shield device pulled back into the aspiration catheter after successful aspiration/removal of thromboembolic material and ready for retrieval from the body.

FIGS. 14A & 14B—Show alternative versions of the shield device.

### DETAILED DRAWINGS DESCRIPTION

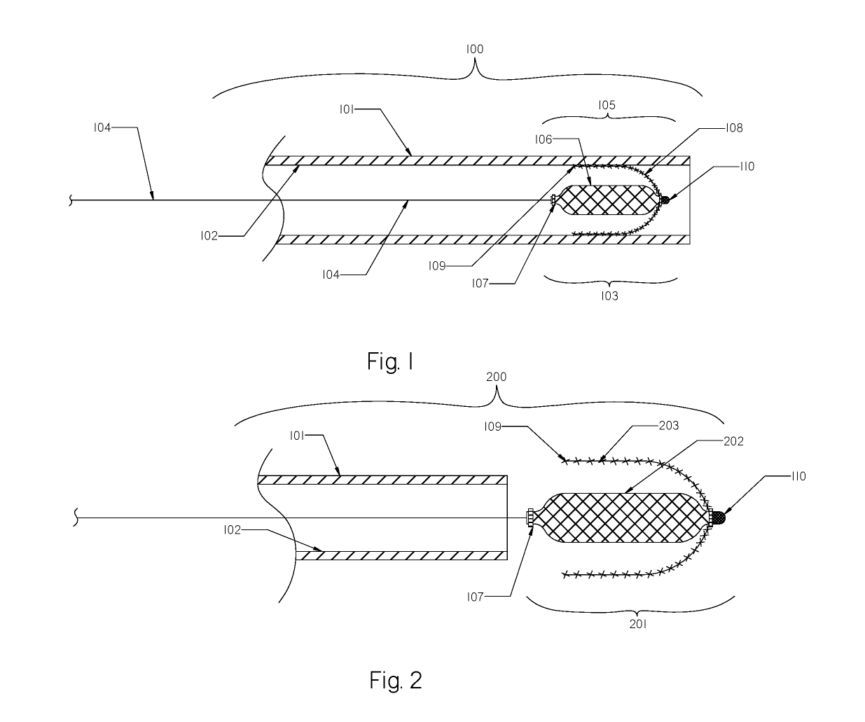

FIG. 1 illustrates a schematic view of the guard device 100 for removal of thromboembolic material from a blood vessel. The guard device 100 comprises the placement catheter 101 having an axial inner lumen 102 and a shield device 103. The shield device 103 comprises a pusher wire 104 and a braid assembly 105. The braid assembly 105 includes an inner expandable braid 106 having a proximal end 107 and an outer expandable braid 108 with an open proximal end 109 and a distal tip 110. The pusher wire 104 is attached to the proximal end 107 of the inner expandable braid 106 using any suitable methods, including but not limited to bonding, gluing, welding, soldering, crimping or other applicable means. The shield device 103 is deliverable through the lumen 102 of the placement catheter 101. The expandable braid assembly 105 is movable during deployment from a first delivery position as shown in FIG. 1 (compressed position) to a second placement position as shown in FIG. 2. In the first delivery position, the expandable braid assembly 105 is in an unexpanded position inside the placement catheter 101 and has a nominal first diameter. In the second position, the braid assembly 105 is in a radially expanded position and has a second nominal diameter which is greater than the first nominal diameter when both the inner expandable braid 106 and the outer expandable braid 108 are deployed within the vasculature of a patient. For better visibility, radiopaque markers may be located on the distal end 110 and on the proximal end 107 of the inner braid 106 (not shown).

The proximal tip 107 of the expandable inner braid 106 connects with the pusher wire 104 and prevents a very proximal end of the inner expandable braid 106 from fully expanding when deployed from the placement catheter 101. The distal tip 110 of the outer expandable braid 108 connects the inner expandable braid 106 and prevents the very distal end of the outer expandable braid 108 and inner expandable braid 106 from fully expanding when deployed from the placement catheter 101. Such distal and/or proximal tips may be made from, but are not limited to, the following materials: metal, polymer, rubber, adhesive or any combination thereof.

During delivery of the guard device 100 to the treatment zones where thromboembolic material is located, the placement catheter 101 is navigated through bends and curves. In such situations, the shield device 103 traverses concomitant bends as the placement catheter 101 when delivered through the placement catheter 101 to the location of the thromboembolic material.

The outer surface of the expandable braid 108 may be covered with any suitable friction reduction polymer, including but not limited to Parylene (poly paraxylylene) or any other suitable polymers, to reduce the friction coefficient to improve ease of deployment and retrieval of the expandable braid assembly 105 into/out of the delivery catheter 101.

FIG. 2 illustrates a schematic view of the guard device 200 (the same guard device 100 is shown in FIG. 1 in unexpanded/compressed configuration) outside the placement catheter 101 with the expanded braid assembly 201 having the inner expandable braid 202 and the outer expandable braid 203 and deployed outside the placement catheter 101. The inner expanded braid 202 (shown in compressed configuration 104 in FIG. 1) and the outer expandable braid 203 (shown in compressed configuration 108 in FIG. 1) may have a preset expanded transverse shape including one of the following configurations: circular, non-circular or a combination of both. The proximal end 109 of the outer expanded braid 203 is free and open, preferably opposed to a blood vessel wall (not shown) to cover a full cross-sectional area of the vessel wall to better capture any dislodged part of the thromboembolic material.

Radiopaque markers may be positioned outside of the braid assembly 201, inside of the braid assembly 201, or in both locations (not shown). Radiopaque markers may also include a radiopaque solder. Alternatively, the expandable braid assembly 105 may include radiopaque components within the expandable braid structure, or braid wires may be made of Nitinol/Platinum composite.

The expanded braid assembly 201 may should have at least 1.5 times larger diameter in its expanded configuration versus its collapsed configuration when inside the placement catheter 101 as shown in FIG. 1. The most common material to make the expandable braid assembly 105/201 is Nitinol or Nickel/Titanium alloy. The expandable braid assembly 105/201 may be formed from a plurality of strands of Nitinol wire having an outside diameter between 0.0005 inches and 0.002 inches, and having a pore size formed between strands in the expanded configuration of less than about 0.5 square mm. The strands of Nitinol wire may have the same diameter or different diameters, and may be formed using circular wire, oval wire, flat wire or any other suitable wire configuration or combinations thereof.

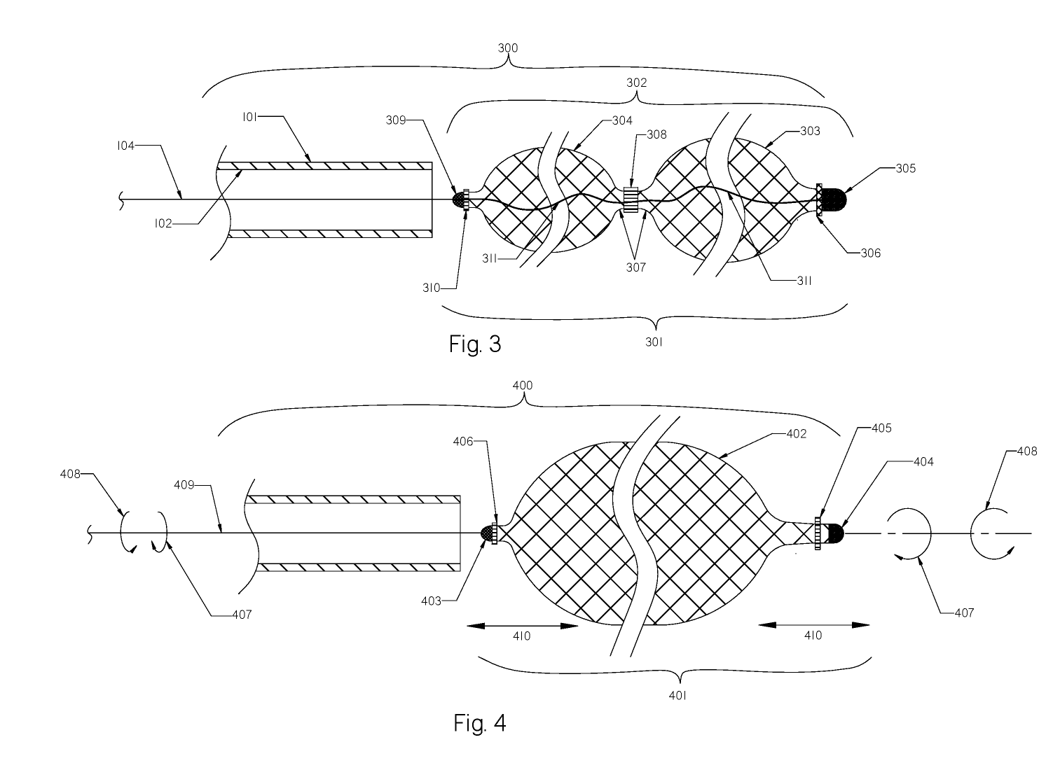

FIG. 3 illustrates a schematic view of an alternative guard device 300 for removal of thromboembolic material from a blood vessel. The guard device 300 comprises the placement catheter 101 having the axial inner lumen 102 and a shield device 301. The shield device 301 comprises the pusher wire 104 and a braid assembly 302 attached together at an attachment area 309. The braid assembly 302 includes a distal tubular larger braid 303 and a smaller proximal braid 304 attached together at an attachment area 307. The braid assembly 302 may be made of one continuous braid shaped accordingly, or it may be made of two different braids attached together. The braid assembly 302 may have a radiopaque marker 306 located on the distal end 305 of the larger braid 303. Another radiopaque marker 308 may be located within the attachment area 307 between the larger braid 303 and the smaller braid 304, and yet another radiopaque marker 310 may be located proximally at the attachment area 309.

The expandable braid assembly 302 attached to the distal end of the pusher wire 104 is deliverable through the inner lumen 102 of the placement catheter 101. The expandable braid assembly 302 is movable during deployment from a first delivery position to a second placement position. The first delivery position of the expandable braid assembly 302 is in an unexpanded position inside the placement catheter 101 and has a nominal first diameter (not shown). The second position of the expandable braid assembly 302 is in a radially expanded position having a second nominal diameter greater than the first nominal diameter when deployed outside the placement catheter 101 and within the vasculature of a patient. The shield device 302 traverses concomitant bends as the placement catheter 101 when delivered through the placement catheter 101 to the treatment location.

The expandable braid assembly 302 may comprise between 8-72 strands made of a monofilament wire having a braid angle of 40 degrees or less in the collapsed configuration inside the placement catheter, and configured to have an expanded braid angle between about 90-150 degrees, and wherein the outside diameter of the expanded braid is between about 1 mm to about 30 mm. The braid assembly 302 may be formed from a plurality of strands having a pore size formed between strands in the expanded configuration of less than about 0.5 square mm.

The expandable braid assembly 302 may comprise between 8-72 strands made of a monofilament wire having a closed pitch of about 5-50 picks per inch in the collapsed configuration inside the placement catheter 101, and when expanded to have 20-100 picks per inch. The expandable braid assembly 302 may have dimensional and material characteristics that result in radial forces on the distal braid 303 when expanded within the vessel. The expandable braid assembly 302 may also have radial force exerted by the expandable proximal braid 304 being close to zero when fully expanded.

An elongate constraining member 311 may be extended at least partially through the expandable braid assembly 302. Such constraining member 311 may connect the distal end 305 of the braid 303 with the proximal end 309 of braid 304 and can be made of material that enhances the radiopacity of the braid assembly 302 by virtue of its composition. Examples of such constraining members include but are not limited to, a wavy platinum coil with inner metal core, radiopaque cable, or any other suitable structure. While the expandable braid assembly 302 shown in FIG. 3 has a tubular shape, other embodiments of the expandable braid assembly 302 may include the distal braid 303 having a tapered configuration to fit vascular configurations that are either tapered distally or tapered proximally (not shown). The expandable braid assembly 302 may have one or more undulations, either partial or along the whole braid configuration (not shown).

FIG. 4 shows another alternative version of the guard device 400 having a shield device 401 comprising a single expandable braid 402 attached to the pusher wire 104 at the proximal end 403 of the expandable braid 402. The radiopaque marker 405 is located on the distal end 404 of the expandable braid 402. Another radiopaque marker 406 is located on the proximal end 403.

The expandable braid 402 is movable during deployment from a first delivery position to a second placement position. The first delivery position of the expandable braid 402 is in an unexpanded position inside the placement catheter 101, and has a nominal first diameter. The second position of the expandable braid 402 is in a radially expanded position having a second nominal diameter greater than the first nominal diameter when deployed outside the placement catheter 101 and within the treatment area or vasculature of a patient. The shield device 401 traverses concomitant bends as the placement catheter 101 when delivered through the placement catheter 101 to the treatment area.

The shield device 401 can be rotated, either through clockwise rotation as shown by arrow 407, anti-clockwise rotation as shown by arrow 408, or a combination of both. Rotation of the shield device 402 may be accomplished by rotating the distal portion 409 of the pusher wire 104. The shield device 401 may also be moved back and forth (as shown by arrow 410) within the treatment area or into and outside the aspiration catheter 101 (not shown). While rotation and back-and-forth movement of the shield device 401 is described in reference to FIG. 4, such rotations and motions may be applied to all shield devices and embodiments of the present invention.

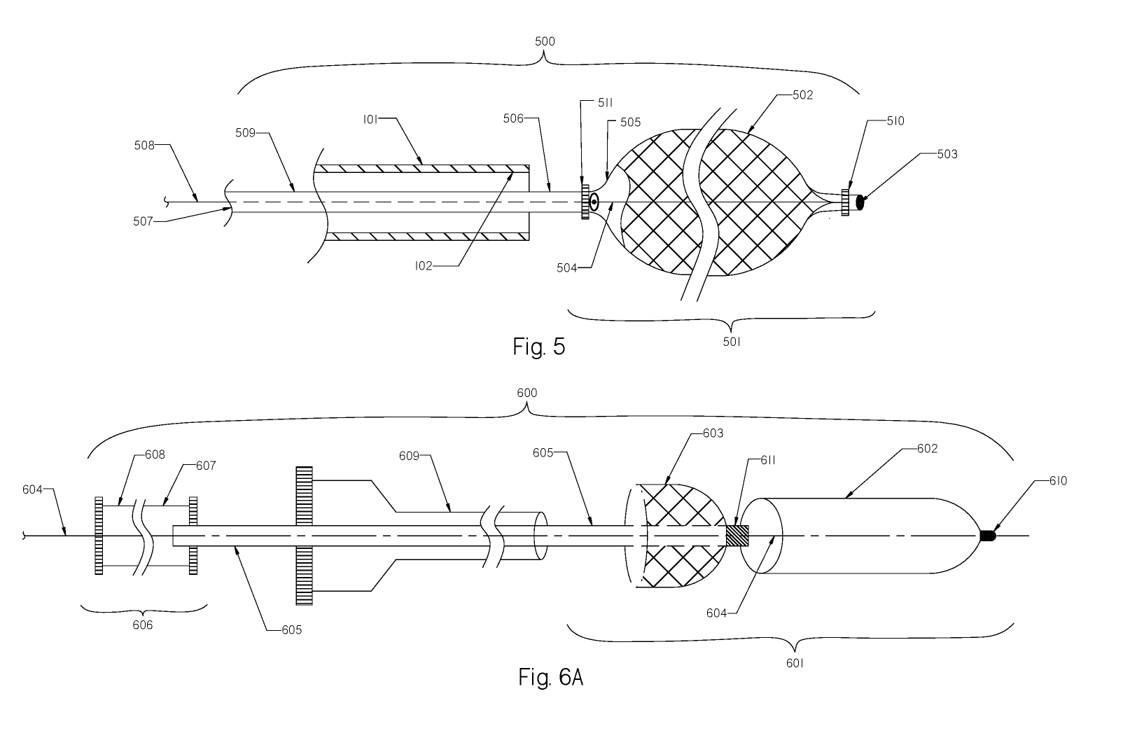

FIG. 5 shows a guard device 500 with a shield device 501 having an expandable braid 502. A pusher wire 504 is attached to the distal end 503 of the expandable braid 502. A pusher tube 506 is attached to the proximal end 505 of the expandable braid 502. The pusher wire 504 is extended within the inner lumen 507 of the pusher tube 506. The pusher tube 506 may be made of polymer, metal or metal alloy in such a way that allows free movement of the pusher wire 504 within the inside lumen 507 of the pusher tube 506. The proximal end 508 of the pusher wire 504 allows the distal end 503 of the expandable braid 502 to be moved distally when the proximal end 508 of the pusher wire 504 is pushed in the distal direction. Thus, while holding the pusher tube 506, the entire expandable braid 502 can be stretched to reduce its outside diameter. This feature may be helpful to ease placement, movement and delivery of the expandable braid 502 through the placement catheter 101 to the treatment location since the inner diameter 102 of the placement catheter 101 is much smaller than the size of the expandable braid 502. The distal end 509 of the pusher tube 506 allows for longitudinal back and forth movement of the proximal end 505 of the expandable braid 502. This feature may be helpful during removal of the expandable braid 502 outside the treatment location. By pushing the proximal end 508 of the pusher wire 504 distally and pulling the proximal end 509 of the pusher tube 506 proximally, the expandable braid 502 will undergo extensive stretching that may be helpful during the deployment of the expandable braid 502 though the placement catheter 101 and its retrieval into the aspiration catheter (not shown).

The shield device 501 with expandable braid 502, attached pusher wire 504 and attached pusher tube 506 are movable during deployment from a first delivery position to a second placement position. The first delivery position of the expandable braid 502 is in an unexpanded position inside the placement catheter 101 and has a nominal first diameter, and the second position of the expandable braid 502 is in a radially expanded position having a second nominal diameter greater than the first nominal diameter when deployed within the treatment area of a patient. The shield device 501 traverses concomitant bends as the placement catheter 101 when delivered through the placement catheter 101 to the treatment location. The shield device 501 is movable distally during deployment using the distally attached pusher wire 502, and retracted proximally using the pusher tube 506. A radiopaque marker 510 may be positioned on the distal end 503 of the expandable braid 502. Another radiopaque marker 511 is positioned on the proximal end 505 of the expandable braid 502.

The shield device 501 may be rotated and repositioned back and forth within the treatment area as desired. By pulling/pushing the pusher tube 506 and pulling/pushing the pusher wire 504, the size of the expandable braid 502 may be adjusted according to clinical need. The proximal portion 505 of the expandable braid 502 when retrieved back into the aspiration catheter (not shown) may provide a plunger or a separator to move blood clots proximally into the aspiration catheter in case the aspiration catheter becomes clogged (not shown).

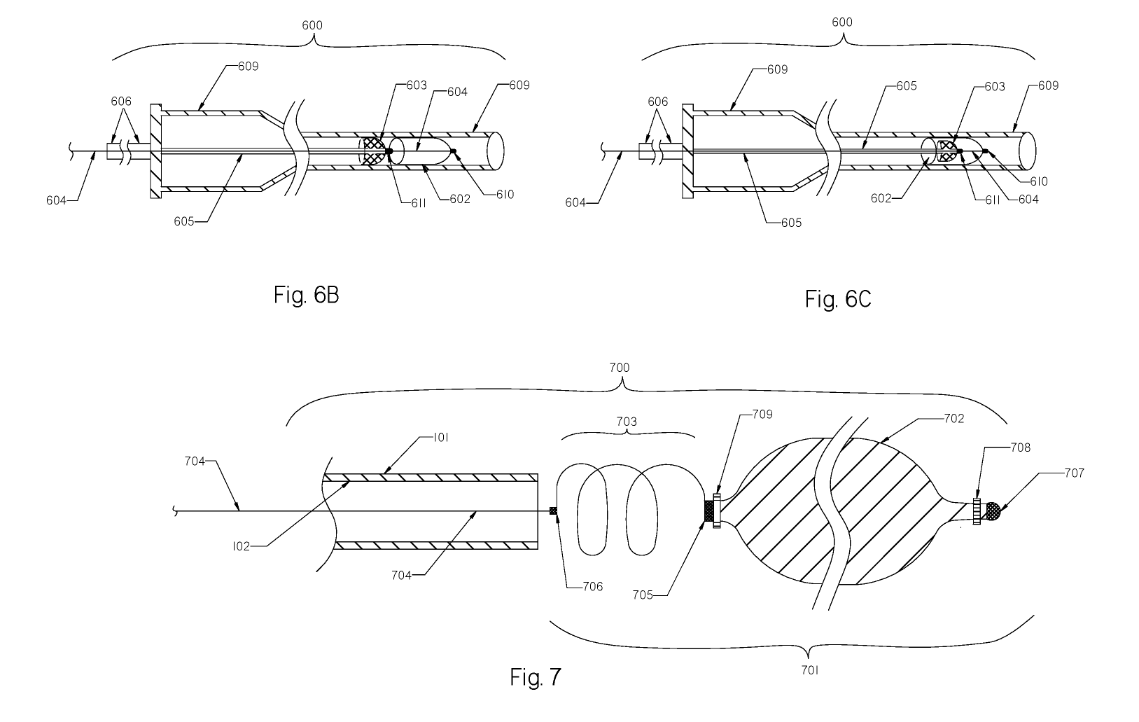

FIG. 6A illustrates a schematic view of an alternative version of the guard device 600 comprising a shield device 601, a placement catheter 609 and Touhy Borst 606. The shield device 601 includes a coaxial retrieval open-ended sleeve 602, an open-ended expandable braid 603, a pusher wire 604 attached to the distal end 610 of the retrieval sleeve 602, and a pusher tube 605 attached to the distal end 611 of the expandable braid 603. The pusher wire 604 attached to the distal end 610 of the retrieval sleeve 602 extends coaxially through a pusher tube 605 that is attached to the distal end 611 of the expandable braid 603. The proximal end of the pusher tube 605 is secured to a double-sided Touhy Borst valve 606. The proximal end of the pusher wire 604 passes through the double-sided Touhy Borst valve 606, and may be selectively clamped down with the distal part 607 of the Touchy Borst 606 holding the pusher tube 605, and the proximal part 608 of the Touhy Borst 606 holding the pusher wire 604. Such a double-sided Touhy Borst connection allows for the pusher wire 604 and the pusher tube 605 to either be manipulated in conjunction with or independently from one another. The double-sided Touhy Borst valve 606 may also be entirely removed from the pusher tube 605 and the pusher wire 604 to facilitate removal of the placement catheter 609.

FIG. 6B shows the guard device 600 with the expandable braid 603 in the collapsed configuration and the attached retrieval sleeve 602 delivered through the placement catheter 609 in a serial fashion. The expanded braid 603 and retrieval sleeve 602 may be further advanced in this manner to the treatment site and through the thromboembolic material.

Alternatively, FIG. 6C shows the guard device 600 with the expandable braid 603 in the collapsed configuration inside the retrieval sleeve 602 delivered through the delivery placement catheter 609 in a parallel overlapping fashion. The system may be further advanced in this manner to the treatment location and through the thromboembolic material.

The expandable braid 603 is movable during deployment from a first delivery position to a second placement position. The first delivery position of the expandable braid 603 is in an unexpanded position inside the placement catheter 609 having a nominal first diameter, and wherein in the second position of the expandable braid 603 is in a radially expanded position having a second nominal diameter greater than the first nominal diameter when deployed outside the placement catheter 609 and within the vasculature of a patient.

The retrieval sleeve 602 incorporates a pusher wire 604 attached to its distal end 610, the pusher wire 604 extends coaxially through the pusher tube 605 that is attached to the distal end 611 of the expandable braid 603 and moves independently. When pulling the pusher wire 604, the expandable braid 603 collapses distally into the proximal open end of the retrieval sleeve 602. The shield device 601 (FIG. 6A) traverses concomitant bends as the placement catheter 609 when delivered through the placement catheter 609 to the treatment location.

FIG. 7 shows an alternative version of the guard device 700 comprising a shield device 701 and the placement catheter 101. The shield device 701 comprises an expandable braid 702 having a distal tip 707 and an expandable plunger or separator 703 attached to the proximal end 705 of the expandable braid 702. The expandable braid 702 has a distal end/tip 707 to prevent the very distal end of the expandable braid 702 from fully expanding when deployed from the placement catheter 101.

The pusher wire 704 is attached to the proximal end 706 of the expandable separator 703. The pusher wire 704 and the expandable separator 703 may be made of two or more components attached together, or may be made from one pre-formed component. When an aspiration catheter (not shown) becomes plugged by clots and is unable to continue aspiration of thromboembolic material, the expandable separator 703 is designated to unplug the aspiration catheter by pulling, pushing and/or rotating the shield device 701 and clots outside and inside of the aspiration catheter. The expandable separator 703 may be rotated to macerate clots inside the plugged aspiration catheter (not shown) and may also engage clots to rotate and further push back and forth, or move inside the aspiration catheter (not shown). The expandable separator 703 shown in FIG. 7 has a helical configuration to illustrate in general a plunger feature or plunger means that can be used to facilitate unclogging of the aspiration catheter when needed. Any suitable configuration of the expandable separator 703 may be considered, including but not limited to a circular structure, a non-circular structure, wire formed wing, looped wires, sinusoidal shape, basket shape, crossing wire shape, and/or a variety of bends. The shield device 701 may have several radiopaque markers 708, 709 placed along the shield device 701 for better visibility. The expandable separator 703 may be made of metal, metal alloys including Nickel-Titanium alloys, polymers or any combination thereof.

While the expandable separator 703 provides a means to un-plug the aspiration catheter in case such clogging of the aspiration catheter occurs, the expandable distal braid 702 provides a distal shield or protection to prevent clot particles or other emboli from moving distally.

The expandable braid 702 and expandable separator 703 are movable during deployment from a first delivery position to a second placement position. The first delivery position of the expandable braid and expandable separator are in an unexpanded position inside the placement catheter 101 having a first nominal diameter. In the second position, the expandable braid 702 and expandable separator 703 are in a radially expanded position having a second nominal diameter greater than the first nominal diameter when deployed from the placement catheter 101 and into the vasculature of a patient. The shield device 701 traverses concomitant bends as the placement catheter 101 when delivered through the placement catheter 101 to the thromboembolic material location.

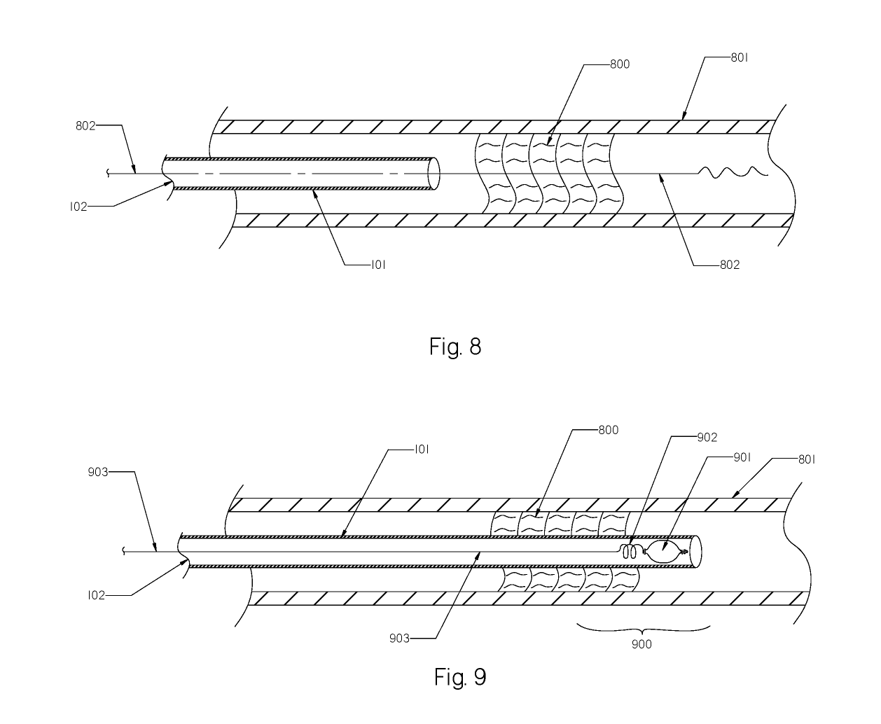

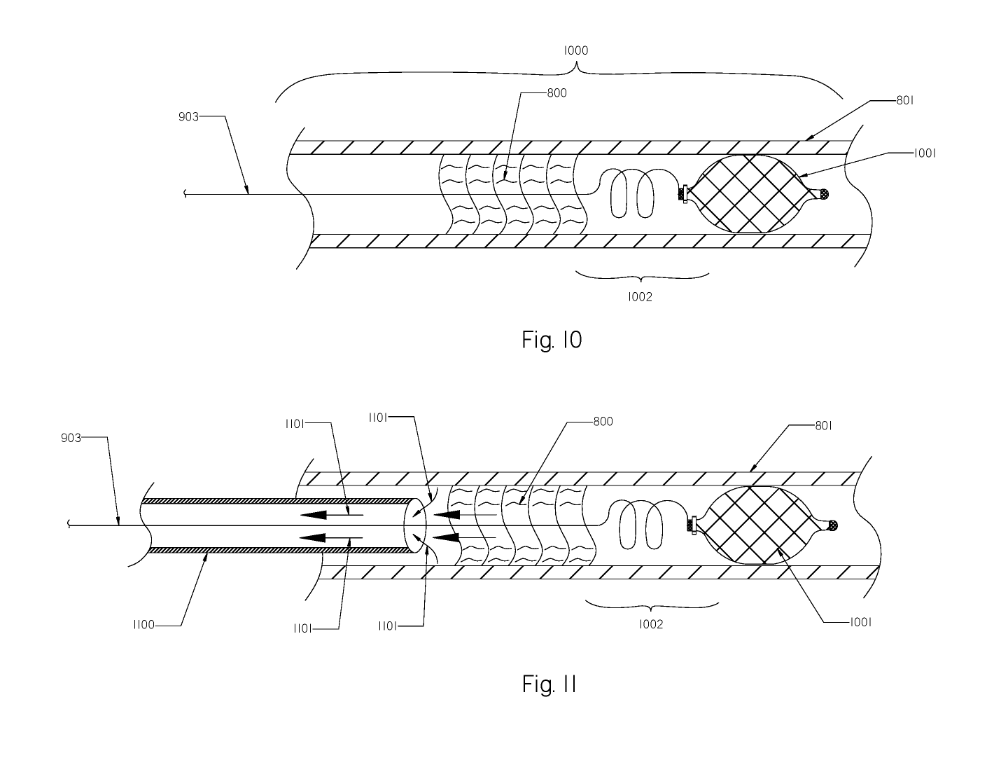

FIGS. 8-13 illustrate how thromboembolic material is removed from a blood vessel according to different methods of the present invention. FIG. 8 shows thromboembolic material or blood clots 800 located inside the blood vessel 801. The placement catheter 101 having the axial lumen 102 is positioned in the vicinity of the blood clot 800. A conventional guide wire 802 is introduced through the placement catheter 101 and crossed though the blood clot 800. If blood clots are soft, a conventional guidewire 802 will easily cross the blood clot 800 as shown. However, if blood clots are older and well organized, the guidewire 802 may go around the blood clot 800 and between blood clots 800 and the vessel wall 801 (not shown). In either case, the distal portion of the guidewire 802 will be placed distally to the location of the blood clot 800. Once the guidewire 802 is positioned beyond the blood clot 800, the placement catheter 101 is pushed through blood clot 800 as shown in FIG. 9. If the guidewire 802 is placed around the blood clot 800, the placement catheter 101 follows the same path (not shown). The placement catheter 101 is a part of the guard device described in the following figures.

Once the placement catheter 101 is positioned across the blood clot 800, the guidewire 802 is removed and the shield device 900 is introduced into the placement catheter 101 as shown in FIG. 9. The shield device 900 can be one of several shield devices described in the present invention, and comprises the expandable braid 901 attached to the expandable separator 902. The expandable braid 901 and the expandable separator 902 are attached proximal to the pusher wire 903. The shield device 900 is pushed through the placement catheter 101 and is intended to be deployed distally beyond the blood clot 800. The placement catheter 101 may be introduced to the treatment area, and location of the blood clot 800, through the aspiration catheter (not shown).

The placement catheter 101 has a sufficient flexibility to navigate the vasculature of the patient and may comprise a proximal end, a distal end and an inner lumen, wherein the inner lumen 102 has a diameter sufficient to receive the expandable braid 900 and the expandable separator 901 in a collapsed unexpanded state, and for advancing the unexpanded braid 901 and unexpanded separator 902 from the proximal end to the distal end of the placement catheter 101. The expandable braid 901 and expandable separator 902 are configured to permit proximal retraction of the braid 901 and the separator 902 into the distal end of the lumen 102 of the placement catheter 101 when the braid 901 and/or separator 902 are partially or fully deployed outside the distal end of the placement catheter 101.

The shield device 1000 shown in FIG. 10 is deployed across the blood clot 800. The expandable braid 1001 is transformed from the unexpanded configuration 901 as shown in FIG. 9 to fully expanded configuration 1001 shown in FIG. 10. Also, the expandable separator 1002 is transformed from the unexpanded configuration 902 shown in FIG. 9 to the fully expanded configuration 1002 shown in FIG. 10.

The expandable braid 1001 and the expandable separator 1002 are movable during deployment from a first delivery position inside the placement catheter 101 to a second placement position outside the placement catheter 101. In the first delivery position, the expandable braid 1001 and the expandable separator 1002 are in an unexpanded position inside the placement catheter 101 having a nominal first diameter. In the second position the expandable braid 1001 and the expandable separator 1002 are in a radially expanded position having a second nominal diameter greater than the first nominal diameter when deployed outside the placement catheter 101.

The expandable braid 1001 may be configured to have a pre-set expanded shape including one of the following configurations: circular shape, non-circular shape or a combination of both. The expanded braid 1001 is configured to assume a radial configuration that opposes the blood vessel wall to prevent the expanded braid 1001 from moving freely along the vessel wall.

In FIG. 11, the aspiration catheter 1100 is introduced over the pusher wire 903 to the location of the blood clot 800. The aspiration catheter 1100 traverses concomitant bends as the pusher wire 903 when delivered to the location of the blood clot 800. The aspiration catheter 1100 may also be positioned over the placement catheter 101 during introduction of the shield device (not shown). When the aspiration catheter 1100 is positioned against the blood clot 800, the aspiration pump (system) is activated as shown by arrows 1101. Aspiration may be provided by any suitable vacuum source including but not limited to: any reusable aspiration pump(s) with suction containers, aspiration wall line in the hospital, or by manual, small disposable liquid vacuum pumps (not shown).

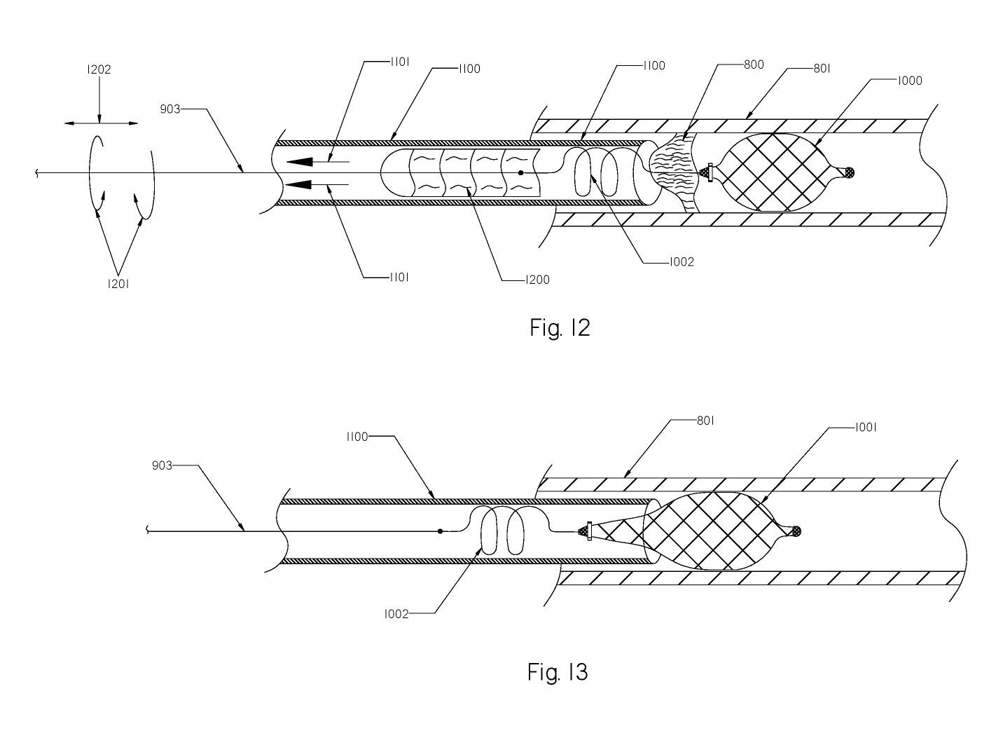

FIG. 12 shows the aspiration catheter 1100 clogged with a portion of the blood clot 1200 partially aspirated in to the aspiration catheter 1100 and too organized or hard to be further aspirated outside the patient. To ease and facilitate blood clot removal and move the portion of blood clot 1200 that blocks the aspiration catheter 1100 distally, the shield device 1000 may be pulled back into the aspiration catheter 1100. When the shield device 1000 is pulled back into the aspiration catheter 1100, the expanded separator 1002 will be first to enter into the aspiration catheter 1100 and force a portion of the blood clot 1200 that is clogging the aspiration catheter 1100 to move proximally. Such pulling of the shield device 1000 is done under aspiration, so the separator 1002 action will disrupt and separate the clogging blood clots 1200 and continue its aspiration outside the patient.

To achieve the same effect of unclogging the aspiration catheter 1100, the aspiration catheter 1100 may be pushed over the pusher wire 903 distally causing the separator 1002 to enter the distal end of the aspiration catheter 1100 and moving the blood clots 1200 proximally. To further facilitate un-clogging of the aspiration catheter 1100, the shield device 1000 may be moved back and forth as desired and rotated clockwise, anticlockwise or both. Such rotations may be done manually, in motorized fashion, or a combination of both.

When the shield device 1000 (pusher wire 903, expandable separator 1002 and expandable braid 1001) is rotated as shown by arrows 1201, the expandable separator 1002 engages the clot material 1200/800 and rotates it inside and/or outside the vessel 801, thereby further unclogging the aspiration catheter 1200, and removing the blood clot or thromboembolic material outside the patient. Longitudinally repositioning of the shield device 1000 as shown by arrows 1202 may provide additional help in moving clot material.

The expandable separator 1002 when retrieved inside the aspiration catheter 1100 exerts pressure against the thromboembolic material 1200/800 in a radially inward direction to facilitate proximal movement of thromboembolic material, thereby preventing the aspiration catheter 1100 from clogging.

The deployed expandable braid 1001 provides distal protection to prevent thromboembolic material from moving distally either after deployment of the shield device 1000 distally beyond the blood clots 800, during the introduction of the aspiration catheter 1200 to the location of the blood clot 800, or during manipulation (rotations and/or forth and back movement) of the shield device 1000 or aspiration catheter 1200 to unclog the aspiration catheter 1200.

The expandable braid 1001 expands inside the vessel 801 to a generally cylindrical shape and may have a size smaller than size of the vessel 801, equal to the size of the vessel, or larger than the size of the vessel. The expandable braid 1001 exerts radial forces on to the vessel wall when expanded to a larger size than the size of the vessel. The expanded braid 1001 expands to a conforming shape as the blood vessel 801 with or without exerting radial forces on to the vessel wall.

To make the blood clot removal process effective and to avoid clogging of the aspiration catheter 1200, the highest possible aspiration pressure should be applied. The clot removal process will be most effective if the process of aspiration pressure build-up time is reduced and/or the maximum aspiration is applied instantaneously.

In the case where the shield device is used without a separator as shown in FIG. 4, the proximal end 403 of the expandable braid 402 provides an identical function as the expanded separator 1002. The aspiration catheter (not shown in FIG. 4) may be advanced against the proximal end 403 of the expanded braid 402 to exert pressure against the thromboembolic material in a radially forward direction to facilitate removal of thromboembolic material and to prevent the aspiration catheter from clogging.

Rotation of the shield device 1000 when the expandable separator 1002 and the expandable braid 1001 are outside the aspiration catheter 1100, or when the expandable separator 1002 is partially inside the aspiration catheter 1100 as shown in FIG. 12, may initiate rotation of the blood clot 800 inside the vessel 801. Such rotation of the blood clot 800 inside the vessel 801 may cause separation of the blood clot mass, and fragmentation and creation of small particles. While the distal flow of small particles will be prevented by the expandable braid 1001, motions of the blood clot 800 within the vessel 800 and/or within the aspiration catheter 1100 may further dismember the blood clot and facilitate its removal outside the patient.

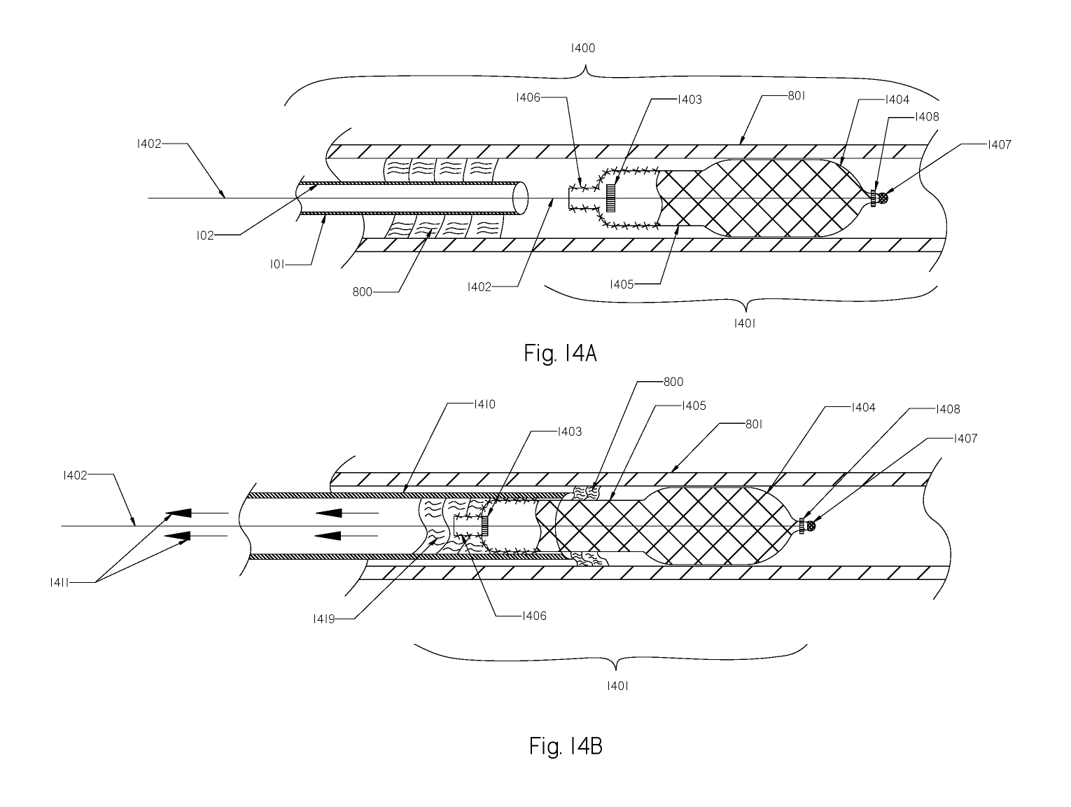

FIG. 13 shows the shield device 1000 (pusher wire 903, expandable separator 1002 and expandable braid 1001) pulled back into the aspiration catheter 1100 after successful aspiration of thromboembolic material from the blood vessel 801.

FIGS. 8-13 illustrate methods and steps to remove thromboembolic material from the blood vessel. While the shield device having the expandable braid 1001 and the expandable separator 1002 was shown in FIGS. 8-13, other shield devices as described in FIGS. 2, 3, 4, 5, 6 can also be used deployed using the same steps and methods. The shield devices used in FIG. 5 and FIG. 6 differ from other shield devices. The shield device shown in FIG. 5 includes the pusher wire 504 attached to the distal end of the expandable braid 502 and the pusher tube 506 attached to the proximal end of the expandable braid 502. This unique shield device structure provides additional attributes with much expanded potential for distal protection and for un-clogging aspiration catheters. The expandable braid 502 may be pushed longitudinally during the introduction of the shield through the placement catheter 101 to the treatment area using the pusher wire 504. The expandable braid 502 may be repositioned and retrieved into the aspiration catheter using the pusher tube 506. The shield device shown in FIG. 6 comprises a pusher tube 605 attached to the distal end of the expandable braid 603. The pusher wire 604 is attached to the distal end of the retrieval sleeve 602. The pusher wire 604 extends coaxially through the pusher tube 605 and moves independently. Pulling the pusher wire 604 proximally collapses the expandable braid 603 into the retrieval sleeve 602.

FIG. 14A shows an alternative version of the shield device 1400 deployed from the placement catheter 101 inside the vessel 801 and positioned distal to the clots 800. The shield device 1400 comprises a braid 1401, a pusher wire 1402 and a stopper 1403 positioned on the pusher wire 1402. The larger distal braid 1404 has a tip 1407 with the radiopaque marker 1408. The puller wire 1402 is attached to the distal braid 1404 at the tip 1407. The braid 1401 comprises a larger distal braid portion 1404 and a smaller proximal braid portion 1405. The proximal braid portion 1405 has a very distal end 1406 that is movable along the pusher wire 1402. The stopper 1403 serves as a stopper preventing the very proximal end 1406 of the proximal braid 1405 from moving distally towards the distal braid 1404 (also known as braid squeezing) when the proximal end 1406 reaches the stopper 1403. The stopper 1403 may be made of polymer, metal or a combination of both and is affixed to the pusher wire 1402 using any suitable attachment methods, including but not limited to gluing, welding, fusing and others. The braid 1401 may be stretched out to fit the inner lumen 102 of the placement catheter 101 when the shield device 1400 is delivered to the treatment site.

FIG. 14B shows the same shield device 1400 as in FIG. 14A. The proximal end 1406 of the braid 1405 is partially retrieved inside the aspiration catheter 1410. During aspiration of the clots 800 into the aspiration catheter 1410 as shown by arrows 1411, a portion 1419 of the clots 800 enters the aspiration catheter 1410 and often clogs the aspiration catheter 1410, preventing clots from being aspirated. To un-clog the aspiration catheter 1410, the braid 1401 is pulled into the aspiration catheter 1410 using the puller wire 1402 such that the proximal end 1406 and the portion 1405 of the braid 1401 enter inside the aspiration catheter 1410. The proximal end 1406 and the proximal braid 1405 exerts pressure against the thromboembolic material 1419 that is clogging the aspiration catheter 1410 in a radially inward direction and facilitates proximal movement of the thromboembolic material outside the patient. The shield device 1400 may be moved back and forth and/or rotated to facilitate movement of the clots 1419 and un-clogging of the aspiration catheter 1410. The stopper 1403 prevents squeezing of the proximal braid 1405 while it is pulled inside the aspiration catheter 1410. Squeezing of the braid 1401, particularly the proximal braid 1405, may result in an increase in its predetermined outside diameter, and present an obstacle towards pulling the proximal braid 1405 inside the aspiration catheter 1410.

To unclog the aspiration catheter 1410 and to move clots 1419 more proximally into the aspiration catheter 1410, the aspiration catheter 1410 may alternatively be pushed distally over the pusher wire 1402 such that the proximal end 1406 and the braid 1405 will enter the aspiration catheter and move the clots 1419 proximally.

The braid 1401 of the shield device 1400 shown in FIG. 14A is deployed distally beyond the clots 800. In an alternative embodiment, the proximal braid 1405 may be at least partially deployed within the clots 800 while the distal braid 1404 is fully deployed distally inside the vessel 801 (not shown).

The device and methods of un-clogging the aspiration catheter shown in FIG. 14A and FIG. 14B have similar functions and operational principles as other devices described: the proximal portion of the shield device serves as a plunger or separator for unclogging the aspiration catheter when needed, while the distal part of the shield device provides distal protection to prevent thromboembolic material from moving distally.

As shown in FIGS. 10-14, the expandable braids of the present invention have a diameter that is at least the same as the diameter of the treatment area, thus providing a proper distal protection function. Preferably, the radial forces of the expandable braid at the treatment area should be at least partially larger than zero. Sizes of the expandable braid may vary, but to facilitate the other function of declogging the placement catheter, the expandable braid should preferably have a diameter that is at least 1.5 times larger in its expanded configuration versus its collapsed configuration when inside the placement catheter.

The present invention is not limited to expandable braids having a uniform number of picks per inch (PPI) of braid or any particular dimensional characteristics. In one embodiment, the braid structure is uniform along the braid length with the same PPI. In alternative embodiments, the braid PPI of the proximal and/or distal end portions are either higher or lower than the PPI in the main body portion of the braid. In one embodiment, the PPI in the proximal portion is higher than those in the main body portion and the distal end of the braid, so that the radial forces exerted in the proximal portion are higher than the radial forces exerted in the main body portion and the distal end of the braid.

The radial strength along the length of the expandable braid may be varied in a few ways. One method is to vary the mass (wire size) along the length of the expandable braid. Another method is to vary the PPI along the length of the expandable braid. The use of higher a PPI will generally provide higher radial forces than those that have lower PPI. Varying the radial force exerted along the length of the expandable braid can be advantageous for use in guarding embolic obstruction so small dislodged particles will not flow distally around the expanded proximal portion of the braid and vessel wall.

Also, the radial force exerted by the expandable braid will reach zero value when the expandable braid is at its designed maximum expandable diameter. The radial forces of the expandable braid at the treatment area should be at least partially larger than zero. Sizes of the expandable braid may be varied, and preferably should have a diameter that is at least 1.5 times larger in its expanded configuration versus its collapsed configuration when inside the placement catheter.

Although the invention has been described above with respect to certain embodiments, it will be appreciated that various changes, modifications, deletions and alterations may be made to such above-described embodiments without departing from the spirit and scope of the invention. Accordingly, it is intended that all such changes, modifications, additions and deletions be incorporated into the scope of the following claims. Drawings and descriptions have been provided that relate to devices and methods for thrombotic material removal from blood vessels with focus on detailed method descriptions related to the expandable separator and expandable braid assembly attached to the pusher wire. However, the scope of the invention includes equally the application of devices and methods that are included in this specification.

1. A guard device for removal of thromboembolic material from a blood vessel, comprising:
a placement catheter having at least one axial lumen;
a shield device comprising a pusher wire with an expandable braid assembly attached to its distal end and deliverable through the lumen of the placement catheter, wherein the expandable braid assembly is movable during deployment from a first delivery position to a second placement position, wherein in the first delivery position the expandable braid assembly is in an unexpanded position inside the placement catheter having a nominal first diameter, and wherein in the second position the expandable braid assembly is in a radially expanded position having a second nominal diameter greater than the first nominal diameter when deployed within the vasculature of a patient, wherein the expandable braid assembly has a distal end with a distal tip at the distal end, where the distal tip prevents the distal end of the expandable braid assembly from fully expanding when deployed from the placement catheter, and
wherein the expandable braid assembly has a diameter that is at least 1.5 times larger in its expanded position than in its collapsed configuration when inside the placement catheter.

2. The device of claim 1, wherein the distal end of the pusher wire is attached to the distal end of the expandable braid.

3. The device of claim 1, wherein the radial forces of the expandable braid when at the treatment area are at least partially larger than zero.

4. The device of claim 1, wherein the expandable braid assembly may be formed from a plurality of strands of Nitinol wire having multiple wire strands of the same dimensions or different dimensions braided into the tubular shape using a circular wire, oval wire, flat wire or any other suitable wire configuration or combination thereof.

5. The device of claim 1, wherein the expandable braid assembly is made of a Nitinol/Platinum composite.

6. The device of claim 1, wherein a friction reduction means is provided on the surface of the expandable braid assembly to improve ease of deployment and retrieval of the placement catheter.

7. The device of claim 1, wherein the expandable braid assembly comprises one or more undulations.

8. The device of claim 1, wherein at least one elongate constraining member is extended at least partially through the expandable braid assembly.

9. The device of claim 1, wherein the expandable braid assembly comprises a proximally tapered section.

10. The device of claim 1, wherein the shield device is configured for rotations.

11. The device of claim 1, wherein the shield device comprises two expandable braids.

12. The device of claim 11, wherein the expandable braid comprises inner and outer expandable braids.

13. The device of claim 1, wherein the expandable braid assembly comprises one continuous braid having at least two different dimensions.

14. A guard device for removal of thromboembolic material from a blood vessel comprises:
a placement catheter having at least one axial lumen;
a shield device comprising an expandable braid having a distal end and a proximal end, a pusher wire attached to the distal end of the expandable braid, a pusher tube attached to the proximal end of the expandable braid and deliverable through the lumen of the placement catheter, wherein the pusher wire extends coaxially through the pusher tube and moves independently thereof, wherein the expandable braid is movable during deployment from a first delivery position to a second placement position, wherein in the first delivery position the expandable braid is in an unexpanded position inside the placement catheter having a nominal first diameter, and wherein in the second position the expandable braid assembly is in a radially expanded position having a second nominal diameter greater than the first nominal diameter when deployed within the vasculature of a patient, and
wherein the braid assembly has a diameter that is at least 1.5 times larger in its expanded configuration than in its collapsed configuration when inside the placement catheter,

15. The guard device of claim 14, wherein the shield device is movable distally during deployment from a first delivery position to a second placement position using the pusher wire.

16. The guard device of claim 14, wherein pulling the pusher tube proximally collapses the expandable braid into the retrieval sleeve.

17. The guard device of claim 14, wherein the shield device is movable distally during deployment from the placement catheter using the pusher tube and the pusher wire together.

18. The guard device of claim 14, further including an expandable separator attached to the pusher wire and the expandable braid.

19. The guard device of claim 14, wherein the expandable braid has a proximal portion adjacent the proximal end thereof, and further comprising a stopper element positioned on the pusher wire to prevent the proximal portion of the expandable braid moving towards the distal end of the shield device.

20. The device of claim 19, wherein the stopper element secures a predetermined proximal braid size when the proximal portion is pushed towards the distal end of the expandable braid while allowing stretching of both the proximal portion and the distal end of the expandable braid when the pusher wire is moved distally.
